(19) United States
(12) Patent Application Publication
(10) Pub. No.: US 2006/0019869 A1
(43) Pub. Date: Jan. 26, 2006

(54) Intradiscal anti-inflammatory therapy involving autologous adiponectin

(76) Inventors: DiMauro M. Thomas, Southboro, MA (US); Attawia Mohamed, Canton, MA (US); Serhan Hassan, South Easton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/938,903

(22) Filed: Sep. 10, 2004

Related U.S. Application Data
(60) Provisional application No. 60/590,526, filed on Jul. 23, 2004.

(51) Int. Cl. A61K 38/38

(52) U.S. Cl. 514/2

(57) ABSTRACT

Administering an effective amount of APN into an intervertebral disc is disclosed.

### CONTINUING DATA

This application claims the benefit of U.S. Provisional Patent Application Ser. No. 60/590,526, entitled “Intradiscal Anti-Inflammatory Therapy Involving Autologous Adiponectin”, DiMauro et al., filed Jul. 23, 2004 (Attorney Docket: DEP5352USASP), the specification of which is incorporated by reference in its entirety.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1β and TNF-α as well as matrix metalloproteinases (MMPs). These cytokines and MMPs help regulate the metabolism of the nucleus pulposus cells.

In some instances of disc degeneration disease (DDD), gradual degeneration of the intervetebral disc is caused by mechanical instabilities in other portions of the spine. In these instances, increased loads and pressures on the nucleus pulposus cause the cells within the disc (or invading macrophases) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

As DDD progresses, toxic levels of the cytokines and MMPs present in the nucleus pulposus begin to degrade the extracellular matrix, in particular, the MMPs (as mediated by the cytokines) begin cleaving the water-retaining portions of the proteoglycans, thereby reducing its water-retaining capabilities. This degradation leads to a less flexible nucleus pulposus, and so changes the loading pattern within the disc, thereby possibly causing delamination of the annulus fibrosus. These changes cause more mechanical instability, thereby causing the cells to emit even more cytokines, thereby upregulating MMPs. As this destructive cascade continues and DDD further progresses, the disc begins to bulge (“a herniated disc”), and then ultimately ruptures, causing the nucleus pulposus to contact the spinal cord and produce pain.

US Published Patent Application No. US2003/0039651 (Olmarker I) teaches a therapeutic treatment of nerve disorders comprising administration of a therapeutically effective dosage of compounds, including inhibitors of MMPs.

In the examples of Olmarker I, Olmarker I further teaches that the therapeutic compounds are to be administered through systemic pathways. In particular, Olmarker I teaches that “the major contribution of TNF-alpha may be derived from recruited, aggregated and maybe even extravasated leukocytes, and that successful pharmacologic block may be achieved only by systemic treatment. Of note, Olmarker I appears to discourage the local addition of at least one therapeutic compound (doxycycline) to an autotransplanted nucleus pulposus to be applied to a spinal cord.

PCT Published Patent Application No. WO 02/100387 (Olmarker II) teaches the prevention of neovasculariation and/or neo-innervation of intervertebral discs by the administration of anti-angiogenic substances. Again, however, Olmarker II teaches systemic administration of these therapeutic agents.

U.S. Pat. No. 6,419,944 (Tobinick I) discloses treating herniated discs with cytokine antagonists. However, Tobinick I teaches that local administration involves a subcutaneous injection near the spinal cord. Accordingly, Tobinick does not teach a procedure involving a sustained delivery of a drug for the treatment of DDD, nor directly administering a drug into the disc.

US Published Patent Application No. 2003/0049256 (Tobinick II) discloses that injection of such therapeutic molecules to the anatomic area adjacent to the spine is accomplished by interspinous injection, and preferably is accomplished by injection through the skin in the anatomic area between two adjacent spinous processes of the vertebral column.

Tobinick II further teaches that the therapeutic compounds may be administered by interspinous injection in the human and that the dosage level is in the range of 1 mg to 300 mg per dose, with dosage intervals as short as two days. Tobinick II further discloses that other therapeutic compounds are administered in a therapeutically effective dose, which will generally be 10 mg to 200 mg per dose, and their dosage interval will be as short as once daily.

Tobinick, Swiss Med. Weekly, 2003, 133, p, 170-7 (Tobinick III) teaches perispinal and epidural administration of TNF inhibitors.

Karppinen, Spine, 28(8), 203, pp. 750-4, teaches intravenously injecting or orally administering infliximab into patients suffering from sciatica.

As with Tobinick I and II, Karppinen does not teach a procedure involving a sustained delivery of a drug for the treatment of DDD, nor directly administering a drug into the disc space.

U.S. Pat. No. 6,352,557 (Ferree) teaches adding therapeutic substances such as anti-inflammatory medications to morselized extra-cellular matrix, and injecting that combination into an interverterbral disc. However many anti-inflammatory agents are non-specific and therefore may produce unwanted side effects upon other cells, proteins and tissue. In addition, the pain-reducing effect of these agents is typically only temporary. Lastly, these agents typically only relieve pain, and are neither curative nor restorative.

Alini, Eur. Spine J. 11(Supp.2), 2002, pp. S215-220, teaches therapies for early stage DDD, including injection of inhibitors of proteolytic enzymes or biological factors that stimulate cell metabolic activity (i.e., growth factors) in order to slow down the degenerative process. Inhibitors of proteolytic enzymes constitutes a broad class of compounds, including i) inhibitors of proteolytic enzyme synthesis and ii) inhibitors of proteolytic enzyme activity. Alini I does not specify any desired types of inhibitors of proteolytic enzymes.

US Published Patent Application US 2002/0026244 (“Trieu”) discloses an intervertebral disc nucleus comprising a hydrogel that may deliver desired pharmacological agents. Trieu teaches that these pharmacological agents may include growth factors such as TGF-β and anti-inflammatory drugs, including steroids. Trieu further teaches that these pharmacological agents may be dispersed within the hydrogel having an appropriate level of porosity to release the pharmacological agent at a desired rate. Trieu teaches that these agents may be released upon cyclic loading or upon resorption.

Goupille, Spine, 23(14), 1998, pp. 1612-1626 identifies Tissue Inhibitors of MMPs (TIMPs) as a degrader of MMP activity. Goupille reports that TIMP-1 and TIMP-2 bind noncovalently to active MMPs in a 1:1 molar ratio and specifically inhibit their enzymatic activity. However, Goupille also identifies corticosteroids, retinoic acid, TGF-β, PGE1 and PGE2 as inhibitors of MMP synthesis; identifies α2-macroglobulin, hydroxamic acid, derivatives, tetracyclines and quinolones as inhibitors of MMP activity, and identifies bFGF, EGF, Retenoic acid, TGF-β, IL-6, IL-1 LIF, dexamethasone, phorbol ester, and synthetic Vitamin A analogs as stimulators of TIMPs. Moreover, as to administration route, Goupille explicitly identifies only the oral administration route.

In sum, although investigators have generally taught the transdiscal administration of inhibitors of proteolytic enzymes, when investigators have specifically identified the administration of highly specific anti-MMPs for spine-related therapy, the investigators appear to teach only the administration to tissue outside the disc (as in Olmarker and Goupille).

Yamamoto, Biochem. Biophys. Res. Comm., 2001, Feb 16, 281(1), 200-5 reports that IL-10 downregulates TGF-β.

### SUMMARY OF THE INVENTION

The present inventors have developed a number of procedures for efficaciously treating degenerative disc disease and sciatica by therapy involving adiponectin (“APN”). In some embodiments, APN is injected in a therapeutic amount into an inflamed joint such as a degenerating disc. In others, APN is combined with viable cells to produce at least one anti-inflammatory compound selected from tissue inhibitor of MMP-1 (IMP-1) and interleukin-10 (IL-10), and the anti-inflammatory compound is then injected.

Therefore, there is provided a method of treating degenerative disc disease in an intervertebral disc of a patient having a nucleus pulposus and an annulus fibrosus, comprising the steps of:
a) intradiscally administering an effective amount of a formulation comprising adiponectin (APN) into the intervertebral disc.

The present inventors have further recognized the multiple benefits afforded by these molecules in treating DDD and sciatica, and so have developed methods of treating DDD involving administering at least one of APN, APN-induced tissue inhibitor of MMP-1 (TIMP-1) and APN-induced interleukin-10 (IL-10) into a degenerating disc or the vicinity of a nerve root.

Therefore, there is provided a method of treating sciatica, comprising the steps of:
a) epidurally administering an effective amount of a formulation comprising APN into the vicinity of a nerve root.

It is believed that APN is strongly anti-inflammatory. In particular, it is believed that APN upregulates certain anti-inflammatory molecules (e.g., TIMP-1, IRAP and IL-10) and downregulates certain pro-inflammatory molecules (e.g., TNF-α, IL-6 and ROS).

The literature appears to recognize the anti-inflammatory nature of APN. Shimda reports that APN has protective actions in the initiation and progression of atherosclerosis through anti-inflammatory and anti-atherosclerotic effects.” Shimada, Clin. Chim. Acta, 2004, Jun 344(1-2):1-12. Yokota indicates that APN is involved in the termination of inflammatory responses, and suggests that APN may have therapeutic applications in diseases caused by excessive inflammatory responses.” Yokota, Blood, 1 Sep. 2000 96(5), 1723-1731. Diez concludes that the ability of APN to increase insulin sensitivity in connection with its anti-inflammatory and anti-atherogenic properties have made this novel adipocytokine a promising therapeutic tool for the future”. Diez, Eur. J. Endocrinology (2003) 148, 293-300.

APN antagonizes TNF-α. Yokota, Blood, 2000, Sep. 1, 96(5), 1723-32 reports that about 10 ug APN/l inhibits phagocytic activity and completely eliminates TNF-α production from LPS-induced phagocytes. In particular, Yokota reported that LPS-induced production of TNF-α in human macrophages dropped from over 800 pg/ml TNF-α to less than 20 pg/ml TNF-α when only 10 ug/l APN was applied. Yokota concluded that APN is an important negative regulator of immune systems, may be a unique suppressor of inflammatory responses because of its specific inhibition of TNF-α transcription, may be involved in ending inflammatory responses, and may have therapeutic applications in diseases casued by excessive inflammation.

Wulster-Radcliffe, Biochem. Biophys. Res. Comm., 316(2004), pp. 924-929, also reports that pretreatment of human macrophages with 10 ug/ml APN suppressed TNF-α production by about 50%, and hypothesized that some of the anti-inflammatory actions thereof are mediated in part by APN suppression of NFκB signaling and ERK1/2 activity.

Therefore, in some embodiments, the APN is directly injected into the disc, preferably to antagonize TNF-α. More preferably, it further antagonizes IL-6 and ROS. Accordingly, there is provided a method of treating degenerative disc disease in an intervertebral disc having a nucleus pulposus and an annulus fibrosus, comprising intradiscally administering an effective amount of a formulation comprising APN into an intervertebral disc.

In some embodiments, the APN may be autologous, while in others it may be exogenous. When exogenous APN is selected, it is preferably recombinant.

In the pathology of sciatica, a portion of the nucleus pulposus (NP) is ejected from the disc and contacts nerve roots adjacent the spinal cord. This provokes an inflammatory response, as macrophages gather around the ejected NP material and release pro-inflammatory cytokines, and TNF-α in particular. It is believed that TNF-α then contacts the nerve root, thereby provoking leg pain.

Since TNF-α is thought to be a primary contributor of leg pain induced by sciatica, and APN antagonizes TNF-α, in some embodiments, an effective amount of APN is injected epidurally near a nerve root in the vicinity of a ruptured intervertebral disc in order to treat sciatica.

APN upregulates TIMP-1. For example, Kumada, Circulation, 2004, May 4, 109(17) 2046-9 reports that APN indirectly increases the concentration of tissue inhibitor of MMP-1 (TIMP-1) through IL-10 upregulation when combined with human macrophage monocytes.

Therefore, in another embodiment, viable cells and APN are cultured ex vivo to produce TIMP-1, and an effective amount of the TIMP-1 is then injected into the disc. In particular embodiments, there is a method of treating inflammation wherein viable cells capable of inducibly expressing TIMP-1 are cultured in the presence of an TIMP-1-inducing agent to produce an effective amount of TIMP-1. The TIMP-1 is then injected into a joint (preferably a degenerating disc). This method is advantageous in that sufficient ex vivo production of TIMP-1 is insured by the clinician's ability to provide as much time as is needed to produce a sufficient quantity of TIMP-1.

Therefore, in accordance with the present invention, there is provided a method of treating degenerative disc disease in an intervertebral disc having a nucleus pulposus and an annulus fibrosus, comprising the steps of:
a) obtaining from the patient cells viable capable of producing TIMP-1;
b) mixing an TIMP-1-inducing composition with the viable cells for a period sufficient to produce TIMP-1, and
c) intradiscally administering an effective amount of a formulation comprising TIMP-1 into the intervertebral disc.

In other embodiments, adiponectin is combined ex vivo with viable cells capable of expressing TIMP-1, and this mixture is then injected into the disc and thereafter produces in vivo an effective amount of TIMP-1 inside the disc. In particular embodiments, there is a method of treating inflammation wherein viable cells that are capable of producing TIMP-1 are mixed with an TIMP-1-inducing agent and then are injected into a joint (preferably a degenerating disc), whereby the inducable cells thereafter produce in vivo an effective amount of TIMP-1. This method is advantageous in that sufficient in vivo production of TIMP-1 is insured by the clinician's ability to provide as many viable cells as is needed to produce an effective amount of TIMP-1. Moreover, since the cells are injected prior to induction, there is no need to wait for an ex vivo incubation period.

Therefore, in accordance with the present invention, there is provided a method of administering TIMP-1 to a patient, comprising:
a) obtaining from the patient cells viable capable of producing TIMP-1;
b) mixing an TIMP-1-inducing composition with the viable cells for a period sufficient to produce induced cells, and
c) administering the induced cells to a location in the patient, whereby the induced cells in vivo produce TIMP-1 at the location.

Wulster-Radcliffe, Biochem. Biophys. Res. Comm., 316(2004), pp. 924-929, also further reports that pretreatment of human macrophages with 10 ug/ml APN suppressed IL-6 production by about 50%, and concluded that the anti-inflammatory properties of APN should extend to negative regulation of IL-6 as well.

Kumada has linked APN to the upregulation of IL-10. Kumada, supra, hypothesizes that APN increases the TIMP-1 level through IL-10 expression.

Wulster-Radcliffe, Biochem. Biophys. Res. Comm., 316(2004), pp. 924-929, also further reports that pretreatment of human macrophages with 10 ug/ml APN induced IL-10 production, and concluded that APN upregulates IL-10.

IL-10 has also been reported to be a potent anti-inflammatory molecule. For example, Cassatella, J. Exp. Med., 1993, Dec. 1, 178(6) 2207-11, reports that IL-10 inhibits the release of pro-inflammatory cytokines. Cassatella, J. Exp. Med., 1994 May 1, 179(5) 1695-9, reports that IL-10 upregulates IRAP in neutrophils.

Since IL-10 is a strong anti-inflammatory produced by APN, the present invention also contemplates the direct injection of APN-induced IL-10 as well. Therefore, in another embodiment, viable cells and APN are cultured ex vivo to produce an effective amount of IL-10, and the IL-10 is then injected into the disc. In particular embodiments, there is a method of treating inflammation wherein viable cells capable of inducibly expressing IL-10 are cultured in the presence of an IL-10-inducing agent (such as APN) to produce IL-10. The IL-10 is then injected into a joint (preferably a degenerating disc). This method is advantageous in that sufficient ex vivo production of IL-10 is insured by the clinician's ability to provide as much time as is needed to produce a sufficient quantity of II-10.

Therefore, in accordance with the present invention, there is provided a method of treating degenerative disc disease in an intervertebral disc having a nucleus pulposus and an annulus fibrosus, comprising the steps of:
a) intradiscally administering an effective amount of a formulation comprising IL-10 into the intervertebral disc.

In other embodiments, adiponectin is combined ex vivo with viable cells capable of expressing IL-10. This mixture is then injected into the disc and thereafter produces an effective amount of interleukin-10 inside the disc. In particular embodiments, there is a method of treating inflammation wherein viable cells that are capable of producing IL-10 are mixed with an IL-10-inducing agent and then are injected into a joint (preferably a degenerating disc), whereby the inducable cells thereafter produce in vivo an effective amount of IL-10. This method is advantageous in that sufficient in vivo production of interferon is insured by the clinician's ability to provide as many viable cells as is needed to produce an effective amount of IL-10. Moreover, since the cells are injected prior to induction, there is no need to wait for an ex vivo incubation period.

Therefore, in accordance with the present invention, there is provided a method of administering IL-10 to a patient, comprising:
a) obtaining from the patient cells viable capable of producing IL-10;
b) mixing an IL-10-inducing composition with the viable cells for a period sufficient to produce induced cells, and
c) administering the induced cells to a location in the patient, whereby the induced cells in vivo produce IL-10 at the location.

It is further believed that APN has anti-oxidant capabilities. Motoshima, Biochem. Biophys. Res. Comm., 315 (2004) 264-172 reports that the global component of APN suppresses cellular superoxide generation in endothelial cells treated with oxidized LDL. Accordingly, it is believed that the application of APN to an inflamed disc would have the further effect of antagonizing the reactive oxygen species (ROS) therein.

Therefore, it appears that APN holds a special advantage as a therapeutic compound in treating DDD in that it not only is a potent anti-inflammatory molecule that can strongly antagonize a key pro-inflammatory lynchpin of DDD (TNF-α), APN also induces the production of two key anti-inflammatory proteins (TIMP-1 and IL-10).

### DESCRIPTION OF THE FIGURES

FIG. 1 is a schematic of an APN separation system.

FIG. 2 is a cross-section of a APN-filled cartridge housed within a syringe.

FIGS. 3a-3e are cross-sections of the use of a bellows-type syringe in accordance with the present invention.

FIGS. 4a and 4b are cross-sections of a syringe of the present invention having a porous annulus adapted for separating APN from plasma.

FIGS. 5a-5b are cross-sections of a syringe of the present invention having a collagen particles therein for separating APN from plasma.

FIGS. 6a-6c are cross-sections of a syringe useful with the present invention.

FIG. 7 is a cross-section of a centrifugation container filled with whole blood.

FIG. 8 is a cross-section of a centrifugation container filled with centrifuged blood.

FIG. 9 is a side view of a syringe having a needle inserted into the container of FIG. 8.

FIG. 10 is a side view of a syringe filled with APN-rich plasma.

FIG. 11 is a cross-section of a syringe of the present invention injecting APN-rich plasma into the nucleus pulposus of an intervertebral disc.

### DETAILED DESCRIPTION OF THE INVENTION

Ouichi, Circulation, 103(8), 2001, Feb. 27, p. 1057 has reported that APN is present in an abundance in whole blood, typically accounting for 0.01% of the protein content of human blood. Therefore, in preferred embodiments, APN is preferably obtained from the whole blood of the patient, as it is present there in large amounts.

In one particularly preferred embodiment, whole blood obtained from the patient is centrifuged to provide a plasma portion. It has been reported by Yokota, supra, that APN levels in plasma of healthy humans ranges from 1.9-17.0 μg/ml. Since it has been reported by Yokota that the inhibitory effect of APN upon TNF-α was seen at about 5 μg/ml, it is noted that unconcentrated plasma may likely possess levels of APN that are therapeutically effective in stopping inflammation in joints.

Therefore, in some embodiments, there is provided a method of treating an inflamed joint, wherein a formulation consisting essentially of plasma comprising an effective amount of APN is injected into the joint, preferably an intervertebral disc space. In some embodiments thereof, an APN-rich portion of the plasma is obtained, for example, by using a gradient fluid with a centrifuge, and then injecting that APN-rich fraction into the disc.

In some embodiments, the plasma may be centrifuged in a container having a plurality of side ports. A needle may be passed through one of these side ports to access the APN-rich layer of the plasma.

In some embodiments, the plasma portion is separated from the remainder of the blood and passed through an affinity column containing a separation material for which APN has a high affinity. The APN is thus preferentially adsorbed onto the separation material. Next, adsorbed APN is eluted from the separation material using a suitable elution solution.

Nakano, J. Biochem (Tokyo), 1996 Oct. 120(4) 803-12, examined methods for isolating APN, and found not only that APN binds specifically to gelatin, but also that it can be eluted from the gelatin material by a 1M NaCl solution. Nakano further reported that applying these methods to 500 ml of human plasma resulted in the isolation of about 50 μg of APN.

Therefore, in preferred embodiments, the plasma portion is separated from the remainder of the blood and passed through an affinity column containing gelatin (or collagen I, III or V), and the adsorbed APN is eluted from the column using a 1 M NaCl solution.

In other embodiments, APN may be separated from the collagen by digesting the collagen with, for example, trypsin or collagenase.

In other embodiments, APN is obtained from adipose tissue, as it is exclusively released by adipose tissue.

In other embodiments, other conventional separation procedures may be used to separate APN from the other components of whole blood or fat.

Once the APN has been isolated, it is, in some embodiments, combined with viable cells either in vivo or ex vivo. These combinations are provided in order to induce production of an effective amount of at least one of IL-10, TIMPs-1, and mixtures thereof.

In preferred viable cell embodiments thereof, a physiologic fluid containing viable leucocyte cells is obtained from the patient. Preferably, the physiologic fluid is derived from whole blood. Whole blood contains monocytes capable of producing IL-10 or TIMP-1 and is easily obtainable from the patient. More preferably, the whole blood is then fractionated by a conventional procedure (such as centrifugation or filtration) to obtain a selected portion of whole blood having a relatively high concentration of monocytes or neutrophils.

In some embodiments, the leucocytes are derived from the buffy coat fraction of whole blood. The buffy coat typically comprises about 5-10 vol % of whole blood Utilization of the buffy coat in the present invention is desirable because it contains a concentrated amount of monocytes capable of producing autologous IL-10 or TIMP-1. Typically, the concentration of monocytes will be on the order of 10-20 fold over that found in whole blood. In some embodiments, a portion of the buffy coat may be used.

In other embodiments, the buffy coat is combined with other portions of blood in order to exploit desirable properties of molecules present in the other portions of blood.

For example, in some embodiments, the buffy coat is combined with at least a portion of the plasma fraction. The plasma fraction contains fibrinogen and so may be useful for clotting the inducing composition to insure that the induced cells that are injected into the disc space remain in the disc space, or for forming a sustained release device for APN, IL-10 or TIMP-1.

In other embodiments, the buffy coat is combined with thrombin in order to produce clotting.

In some embodiments, the buffy coat is combined with at least a portion of the platelet fraction of the blood. The platelet fraction contains growth factors such as TGF-β, which, upon release, can help stimulate extra cellular matrix production by natural disc cells.

In some embodiments, the viable cells are chondrocytes, and are preferably disc cells. In some embodiments, chondrocytes are conveniently obtained from the patient's tissue by first performing a biopsy upon the patient. In one example, a biopsy is performed using a biopsy punch and results in the removal of about a 5 mm×5 mm×5 mm portion of cartilage, from, for example, an ear. This tissue sample is then subjected to collagen removal (by, for example, adding trypsin to the biopsy tissue) in order to free up the cellular components from the extracellular matrix. The cellular fraction is then subjected to centrifugation in order to concentrate the chondrocyte fraction from other cellular components. The fibroblast isolation process of biopsy-collagen removal-centrifugation can generally take about 3-4 hours.

Conventional protein production technology may be exploited to include a number of unit processes designed to partially purify the concentration of APN. Such conventional processes include the use of glass beads to capture the APN; the use of a 10 kD filter to capture the APN; the use of a molecular sieve to dewater the plasma; the use of ammonium sulfate to precipitate out the APN; and the use of ethanol extraction to precipitate out the APN.

It is reasonable to expect that adoption of at least one of the partial purification techniques described above will lead to a 5-10 fold increase in the APN concentration in the partially purified solution.

It is believed that as little as about 5 μg/ml APN is an effective anti-inflammatory concentration. Greater amount are generally believed to produce greater anti-inflammatory effects.

Accordingly, in some embodiments of the present invention, the formulation comprises at least 5 μg APN/ml, preferably the formulation comprises at least 10 FIG. APN/ml, more preferably at least 20 μg/ml, and more preferably at least 30 μg APN/ml.

In some embodiments, the APN or induced cells may be combined with a sustained release device in order to insure a continued presence of the APN or cells in the inflamed joint. In some embodiments, autologous cryoprecipitated fibrinogen is used to make the sustained release device. Cryopreciptated fibrin may be used not only as a carrier for APN, but also as a structural element that restores some of the natural mechanical functions of the nucleus pulposus. In one embodiment, cryoprecipitated fibrinogen is taken from the patient's blood (that could be donated before surgery or even collected during surgery with a Cell-Saver). Some surgeons are currently using the cryoprecipitate for scar prevention after posterior lumbar surgery. With autologous fibrin, there would be no risk of rejection since the fibrin is from the patient's own blood proteins. The addition of thrombin to the cryoprecipitate creates a stable gel that may have the mechanical properties desirable in a nucleus pulposus replacement material. With time, the disc may replace the cryoprecipitated fibrin with a fibrocartilage-like material, similar to that of the host tissue

Therefore, in accordance with the present invention, there is provided a method of treating degenerative disc disease in an intervertebral disc having a nucleus pulposus and an annulus fibrosus, comprising intradiscally administering an effective amount of a formulation comprising a APN and fibrin glue having a fibrinogen concentration of at least 10 mg/ml.

In some embodiments, adjunct materials disclosed in U.S. patent application Ser. No. 10/631,487, filed Jul. 31, 2003, “Transdiscal Administration of Specific Inhibitors of p38 Kinase” (DEP5144), the specification of which is incorporated by reference in its entirety, are provided along with the APN.

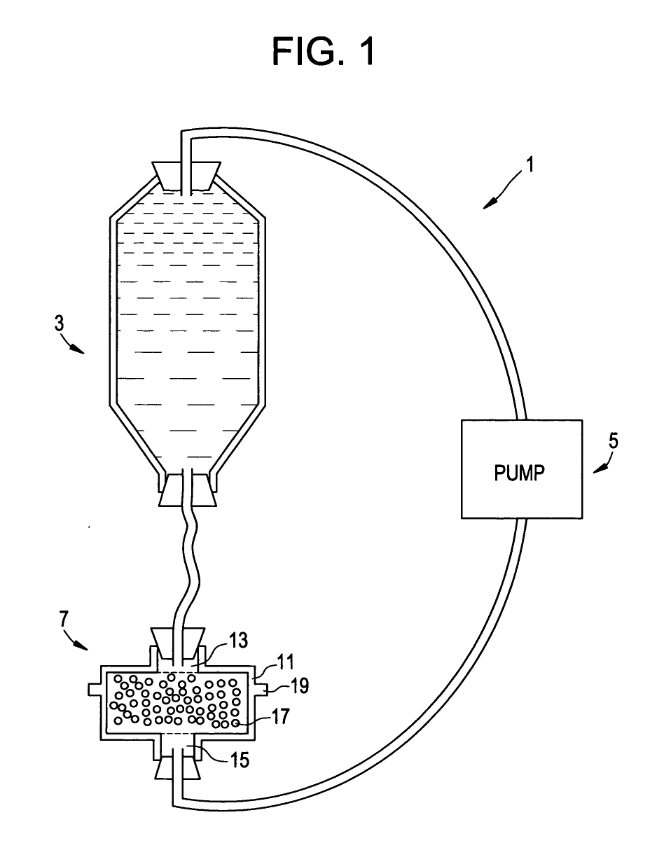

In some embodiments, the affinity column used to isolate APN is provided as a cartridge. Now referring to FIG. 1, there is provided a separation system I for isolating APN, comprising a blood or plasma receptacle 3, a pump 5 and a separation cartridge 7. The separation cartridge comprises a housing 11 having a first open end 13, a second open end 15, and an affinity material 17 housed therein. In some embodiments, the cartridge also has flanges 19 extending from the housing.

In use, blood or plasma is pumped in a closed circuit from the receptacle through the cartridge, through the pump and back to the receptacle. Once the APN has been sufficiently isolated upon the affinity material, the cartridge is removed from the separation system.

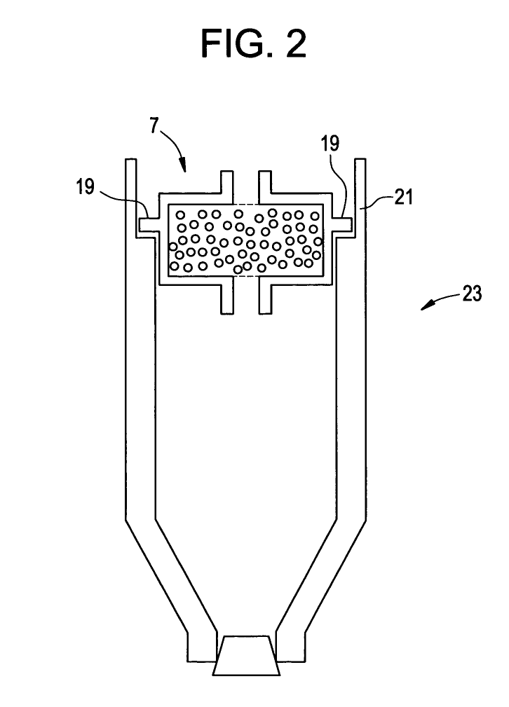

In order to transfer the APN to a syringe for injection, in some embodiments, the cartridge 7 containing the APN is fitted to the proximal end portion 21 of a syringe 23, as in FIG. 2. In this particular embodiment, the flanges 19 of cartridge 7 fit into mating recesses within syringe 23.

Therefore, in accordance with the present invention, there is provided a kit for collecting and administering adiponectin, comprising:
i. a syringe having a barrel having an inner wall, a proximal open end and a distal open end, and
ii. a cartridge having a barrel having an inner wall defining a housing, an outer wall, a proximal open end and a distal open end, wherein the outer wall of the cartridge is adapted to fit within the inner wall of the syringe, and

an adiponectin-binding agent disposed within the housing.

The APN is then eluted into the syringe with a suitable elutant.

Therefore, in accordance with the present invention, there is provided a syringe having a barrel having an inner wall, a proximal open end and a distal open end, wherein the barrel contains an effective amount of adiponectin.

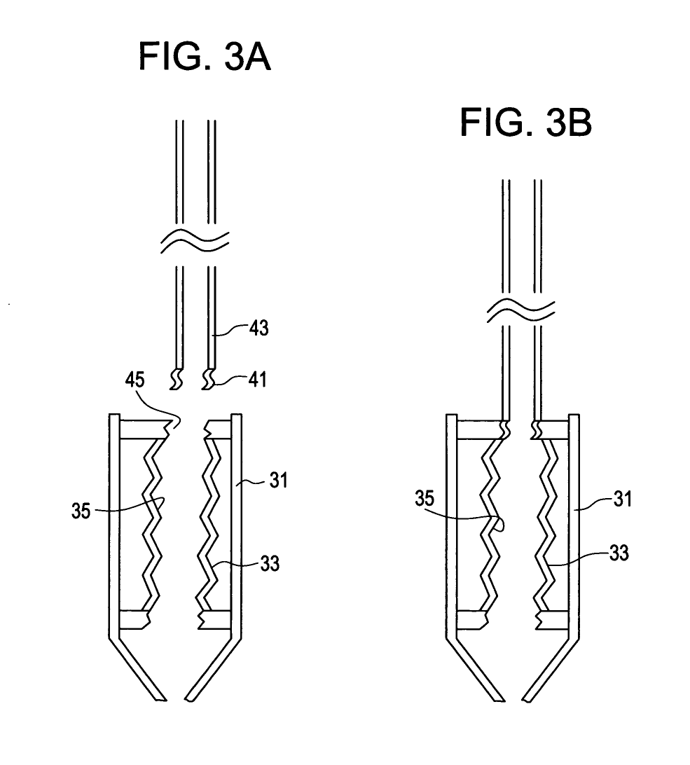

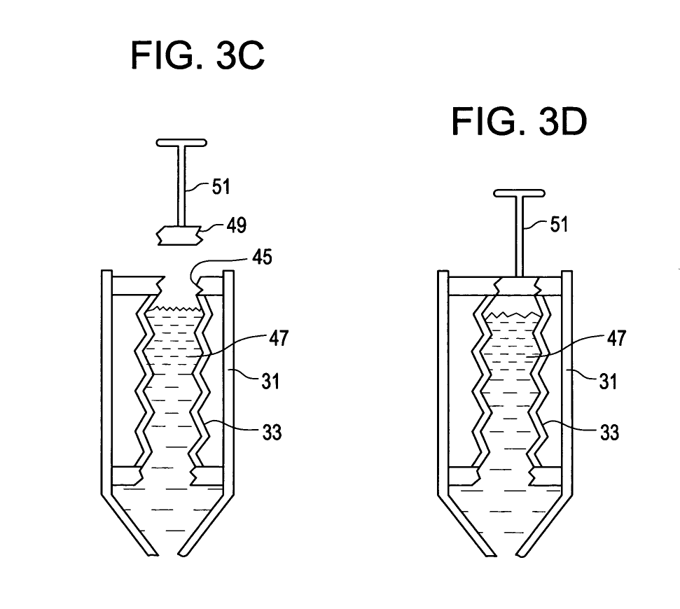

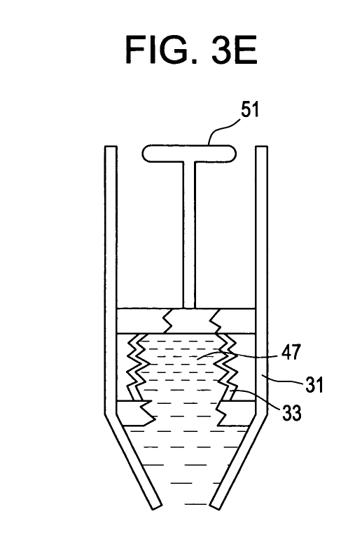

If the isolated APN is to be injected directly into the disc, then, now referring to FIGS. 3a-3e, in some embodiments, a device may be used that is adapted to be both a cartridge in the isolation step and a syringe for the injection step. In one embodiment, the device is designed as a conventional syringe 31 modified with a bellows 33. The inner surface 35 of the bellows is coated with the separation material (not shown) (preferably, collagen I, III or V or a gel Cellulofine™) having a high affinity for APN. Now referring to FIGS. 3a-3b, during the APN separation step, a threaded end 41 of a tubing 43 is threaded into the proximal threaded end 45 of the bellows in order to obtain a secure fitting. Blood or plasma is then passed from the tubing into the bellows, wherein the APN preferentially adsorbs onto the separation material. Now referring to FIG. 3c, after APN isolation step is complete, the tubing is disengaged, and the bellows is filled with an elutant 47 (preferably, a 1M NaCl solution) to elute the APN. Next, and now referring to FIGS. 3c-3d, the threaded plug 49 of plunger 51 is mated to the threaded end 45 of the bellows. Lastly, and now referring to FIG. 3e, plunger 51 is depressed and the eluted APN within bellows 33 is expelled from syringe 31 and into the disc.

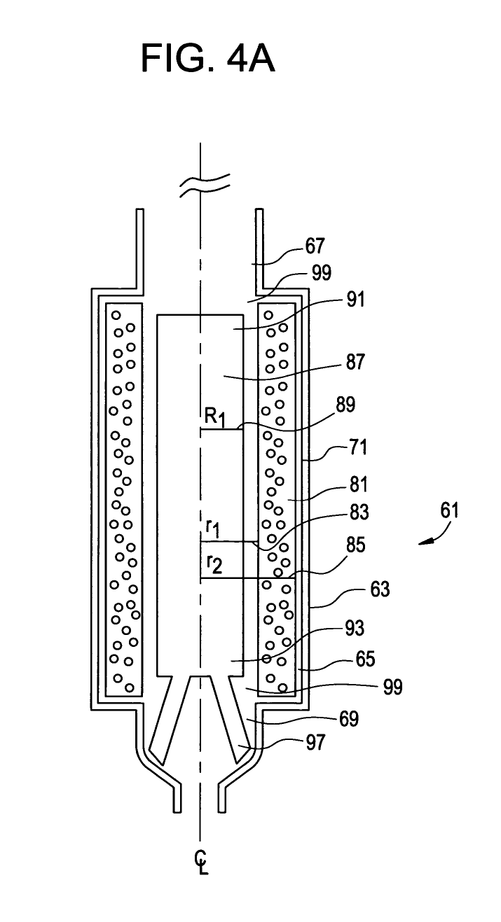

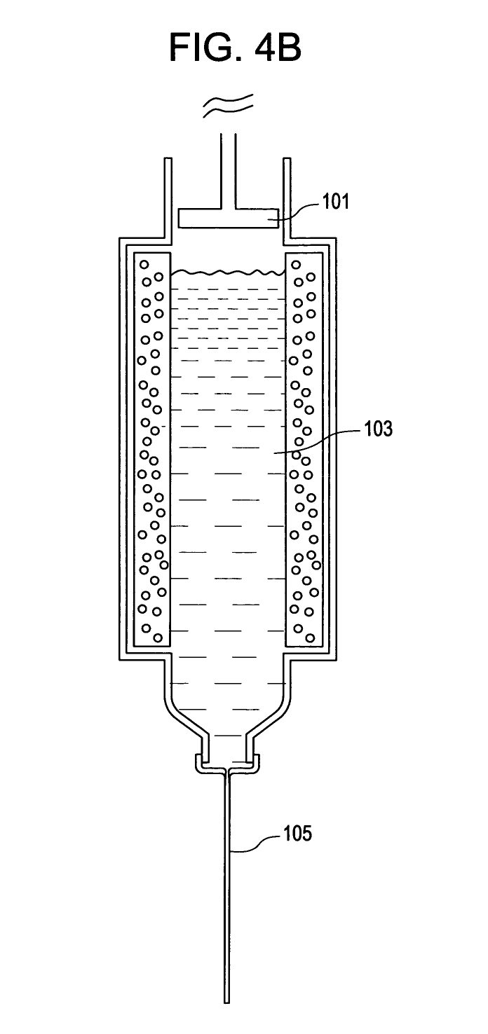

Now referring to FIG. 4a, in some embodiments, the syringe may contain an annulus of collagen adapted for collecting APN. In particular, the apparatus 61 comprises:
a) a syringe 63 comprising a barrel 65 defining a proximal open end 67, a distal open end 69, and an inner wall 71,
b) a porous annulus 81 having an inner radius r₁ 83 and an outer radius r₂ 85, annulus 81 adapted to be received within the inner wall of syringe 63 and collect APN, and
c) a solid plug 87 having an outer radius R₁ 89, a proximal end 91 and a distal end 93, and adapted to be received within the inner radius of the porous annulus 81, and having a plurality of legs 97 extending from the distal end thereof.

In use, the plasma from the patient is run through the syringe of FIG. 4a. Since the length of the plug is less than the length of the annulus, an opening 99 is formed at each end of the barrel for convective plasma flow through the annulus.

Preferably, the porous annulus comprises of a material selected from collagen I, collage III, and collagen V, and mixtures thereof.

In some embodiments, monoclonal antibodies may be used to separate the adiponectin from the rest of the plasma.

After the APN is concentrated in the porous annulus, a small amount of an elutant is passed through the annulus in order to re-solubilize the APN. Preferably, the elutant is 1M NaCl.

Lastly, now referring to FIG. 4b, solid plug 87 is removed and replaced with a plunger 101. When plunger 101 is depressed, APN-rich salt solution 103 is passed distally through syringe 63 and into needle 105 for delivery to the inflamed location.

Therefore, in accordance with the present invention, there is provided an apparatus comprising:
a) a syringe comprising a barrel defining a proximal open end, a distal open end, and an inner wall,
b) a porous annulus having an inner radius and an outer radius, the annulus adapted to be received within the inner wall of the syringe and collect APN.

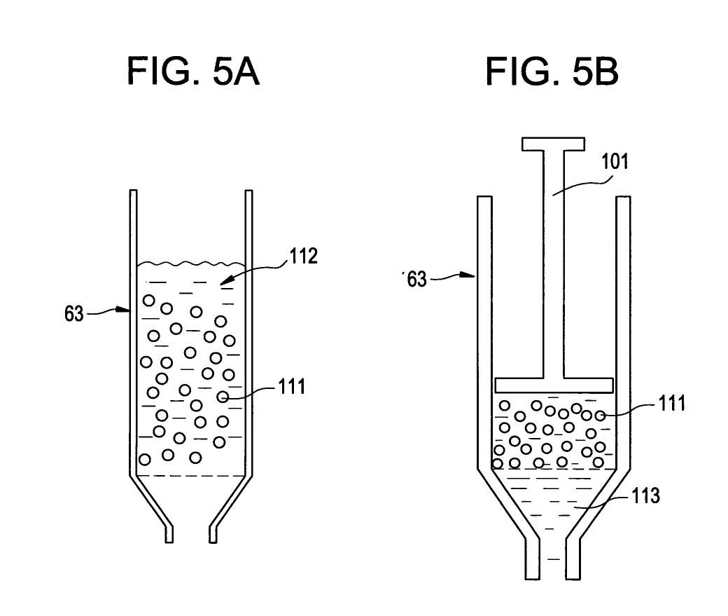

In other embodiments, the and now referring to FIGS. 5a-5b, the porous annulus 81 and solid plug 87 are replaced with collagen particles 111. Plasma or blood 112 is passed through the particles 111 and APN in the plasma attaches thereto. After re-solubilization of the APN with NaCl, a plunger 101 is then placed in syringe 63 and depressed. The spongy nature of collagen particles 111 allows them to be squeezed and displaced distally, thereby pushing the APN-rich solution 113 passed the collagen and through the distal end of syringe 63.

Preferably, a mixing container is used to mix the APN and viable cells, and is adapted to provide homogeneous mixing of the APN and viable cells. In some embodiments, the container is a syringe. In other embodiments, the container is a column having a stopcock.

In embodiments in which collagen particles are used as an affinity column to separate out APN from blood or plasma, it may be desirable to simply forgo the elution step and inject both the collagen particles and the adhered APN into the patient. This procedure has advantage in that the time-consuming elution step is eliminated and the loss and/or dilution of the APN necessitated by the eluant is eliminated. In addition, the collagen particles that are injected may help stabilize the nucleus pulposus portion of the disc and may provide the basis for forming a scaffold for tissue ingrowth.

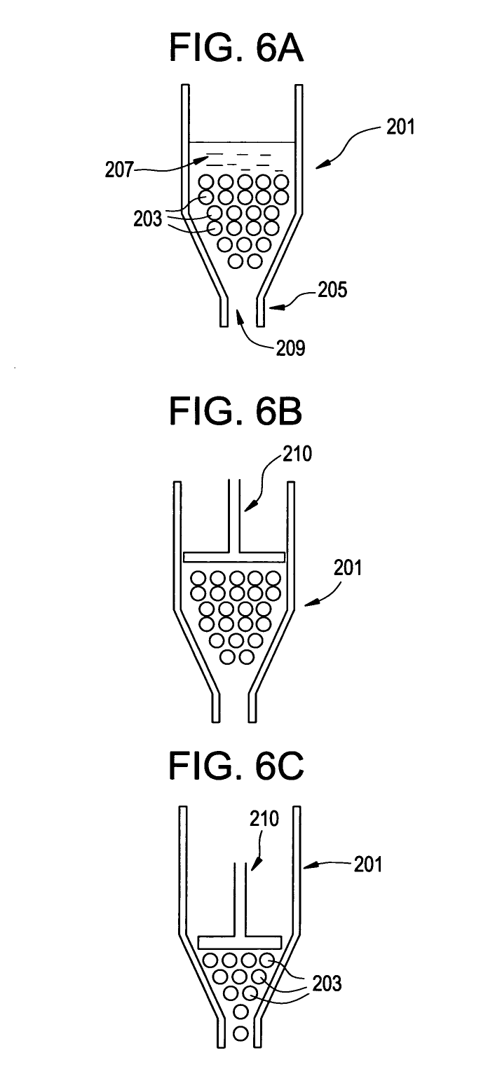

Now referring to FIG. 6a, there is provided a syringe 201 having collagen particles 203 and blood or plasma 207 therein. In this FIG. 6a, the average diameter of the collagen particle 203 is such that is can pass through the opening 209 in the distal end 205 of syringe 201. In this particular case, the average particle size appears to be a little more than one-half the diameter of the distal opening, so that a single particle may pass through the opening, but two at a time may not. Accordingly, blood or plasma 207 may pass through the distal opening 208, but the collagen particles 203 will quickly clog the opening.

Now referring to FIG. 6b, after the plasma has been run through the syringe and APN has been adhered to the collagen particles, a plunger 210 is inserted into the syringe.

Now referring to FIG. 6c, an axial force is applied to plunger 210 to force the collagen particles 203 having APN adhered thereto out of the syringe and into the patient.

In some embodiments, a dewatering agent such as a molecular sieve is provided as a coating upon a substrate. In some embodiments, the substrate can be an inner wall of a syringe or column. In others, the substrate may be in the forms of beads, such as glass or hydroxyapatite beads. In others, the substrate is organic and may be selected from agarose, hyaluronic acid and cellulose acetate.

Because, in some embodiments, the induced cells or APN are immediately injected into the patient so that the patient serves as the incubation receptacle for the induced cells, there is no need to wait for ex vivo production of IL-10 or TIMP-1. Accordingly, in preferred embodiment, the induced cells are injected into the disc less than 10 hours after the mixing step, more preferably less than 5 hours, more preferably less than three hours.

As the injection location is typically inflamed and has a local concentration of IL-10, the APN or induced cells preferably produces an effective amount of IRAP to generate a local in vivo IRAP: IL-10 ratio of at least 1000:1, more preferably at least 10,000:1 (as measured on a molar basis).

Preferably, the APN or induced cells produced in the present invention are injected into an inflamed joint within the patient in a therapeutically effective amount. In some embodiments, the joint is a hip joint. In others, it is a knee joint. In others, it is an intervertebral disc. When the APN or induced cells are injected into an intervertebral disc, they are either injected into the nucleus pulposus, the annulus fibrosus, or both, in order to treat low back pain. In other embodiments, the APN or induced cells are injected epidurally near a nerve root in the vicinity of a ruptured intervertebral disc in order to treat sciatica.

In some embodiments, the APN is injected or produced in an amount effective to reduce or eliminate inflammation present within the local tissue. In others, the APN is injected or produced in an amount effective to act upon nerve endings present within the local tissue and thereby reduce or eliminate pain.

In some embodiments, the APN is injected or produced in an amount effective to reduce or eliminate inflammation and/or pain present within the spinal cord.

In some embodiments, the APN is injected or produced in an amount effective to reduce or eliminate inflammation and/or pain present within a nerve root.

In some embodiments, the APN is injected or produced in an amount effective to reduce or eliminate inflammation associated with Alzheimer's disease within brain tissue.

In some embodiments, APN is coated upon the outside of a hydrocephalus shunt, preferably upon the ventricular catheter.

In some embodiments, APN is used (either as a coating upon a plug or per se) in repair of a hole or tear in an annulus fibrosus.

### EXAMPLE I

This prophetic example describes a typical method of the present invention.

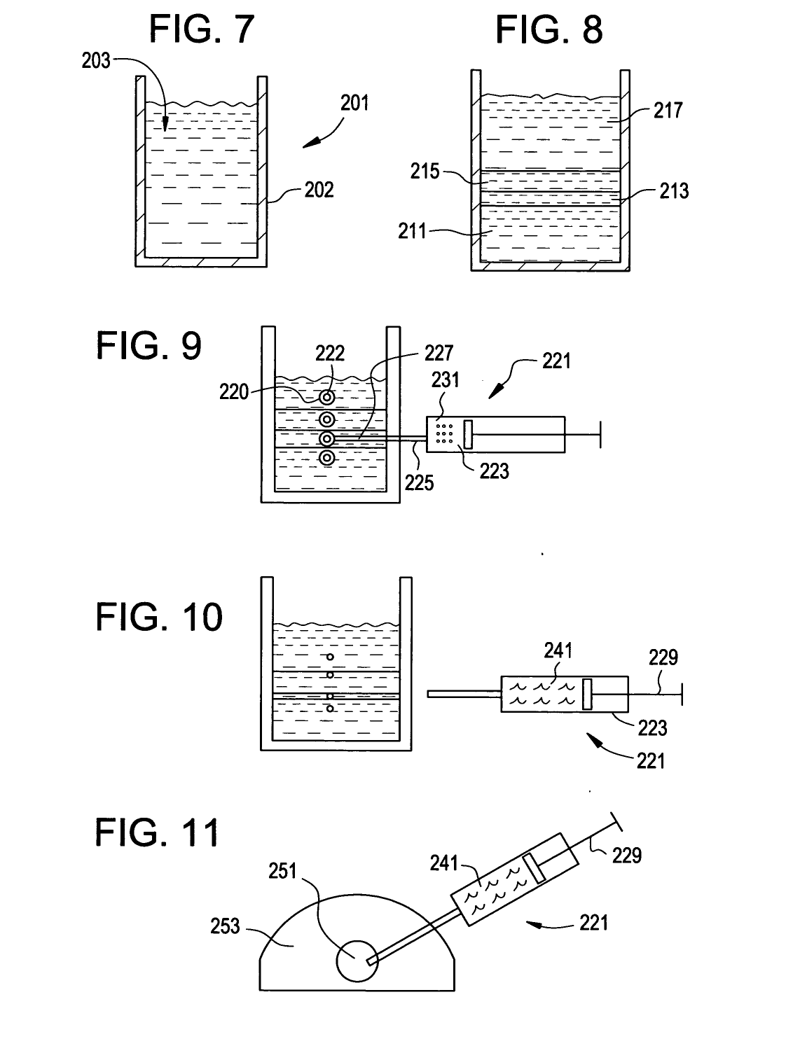

First, about 20 cc of blood 203 is taken from the patient. Now referring to FIG. 7, the blood is placed in a centrifugation container 201 adapted for centrifugation and having a side wall 202.

Now referring to FIG. 8, the blood 203 is centrifuged by a conventional method to produce centrifuged blood fractions including red blood cells 211, platelets 213, buffy coat 215 and platelet poor plasma 217.

Now referring to FIG. 9, a syringe 221 having a barrel 223 and a needle 225 is provided. The centrifugation container has a plurality of side ports 220 having puncturable gaskets 222 therein. The clinician inserts the distal end 227 of the needle through a gasket.

Now referring to FIG. 10, the clinician pulls back upon the plunger 229. The vacuum created by withdrawl of the plunger causes the APN-containing fluid to enter the barrel 223 of the syringe 221. Molecular sieve beads contained within the barrel of the syringe pull water from the fluid, thereby creating APN-rich fluid 241.

Next, the clinician uses a diagnostic test to verify that a particular disc within a patient has high levels of the particular interleukin-1β pro-inflammatory cytokine, MMP-1 or TNF-α.

Next, the clinician provides a local anesthetic (such as 5 ml lidocaine) to the region dorsal of the disc of concern to reduce subcutaneous pain.

Next, the clinician punctures the skin of the patient dorsal the disc of concern with a relatively large (e.g., 18-19 gauge) needle having a stylet therein, and advances the needle through subcutaneous fat and dorsal sacrolumbar ligament and muscles to the outer edge of the intervertebral disc.

Next, the stylet is removed from the needle.

Next, the clinician receives the syringe having the inducing composition of the present invention. This syringe has a smaller gauge needle adapted to fit within the larger gauge needle. This smaller needle is typically a 22 or 24 gauge needle. The barrel of the syringe contains the formulation of the present invention.

Next, the physician advances the smaller needle co-axially through the larger needle and past the distal end of the larger needle, thereby puncturing the annulus fibrosus. The smaller needle is then further advanced into the center of the nucleus pulposus. Finally, and now referring to FIG. 11, the clincian depresses plunger 229 of syringe 221, thereby injecting between about 0.5 and 1 ml of the formulation comprising an effective amount of APN 241 into the nucleus pulposus 251 of the intervertebral disc 253.

We claim:

1. A method of treating degenerative disc disease in an intervertebral disc of a patient having a nucleus pulposus and an annulus fibrosus, comprising the steps of:
a) intradiscally administering an effective amount of a formulation comprising adiponectin (APN) into the intervertebral disc.

2. The method of claim 1 wherein the adiponectin is autologous.

3. The method claim 1 wherein the adiponectin is obtained from the patient's blood.

4. The method of claim 1 wherein the adiponectin is exogenous.

5. A method of treating sciatica, comprising the steps of:
a) epidurally administering an effective amount of a formulation comprising adiponectin (APN) into the vicinity of a nerve root.

6. The method of claim 1 wherein the adiponectin is autologous.

7. The method claim 1 wherein the adiponectin is obtained from the patient's blood.

8. The method of claim 1 wherein the adiponectin is exogenous.

9. A method of treating degenerative disc disease in an intervertebral disc having a nucleus pulposus and an annulus fibrosus, comprising intradiscally administering an effective amount of a formulation comprising a APN and fibrin glue having a fibrinogen concentration of at least 10 mg/ml.

10. The method of claim 9 wherein the fibrin glue has a fibrinogen concentration of at least 20 mg/ml.

11. A method of treating degenerative disc disease in an intervertebral disc having a nucleus pulposus and an annulus fibrosus, comprising the steps of:
a) obtaining from the patient cells viable capable of producing TIMP-1;
b) mixing an TIMP-1-inducing composition with the viable cells for a period sufficient to produce TIMP-1, and
c) intradiscally administering an effective amount of a formulation comprising TIMP-1 into the intervertebral disc.

12. A method of administering TIMP-1 to a patient, comprising:
a) obtaining from the patient cells viable capable of producing TIMP-1;
b) mixing an TIMP-1-inducing composition with the viable cells for a period sufficient to produce induced cells, and
c) administering the induced cells to a location in the patient, whereby the induced cells in vivo produce TIMP-1 at the location.

13. A method of treating degenerative disc disease in an intervertebral disc having a nucleus pulposus and an annulus fibrosus, comprising the steps of:
a) intradiscally administering an effective amount of a formulation comprising IL-10 into the intervertebral disc.

14. A method of administering IL-10 to a patient, comprising:
a) obtaining from the patient cells viable capable of producing L-10;
b) mixing an IL-10-inducing composition with the viable cells for a period sufficient to produce induced cells, and
c) administering the induced cells to a location in the patient, whereby the induced cells in vivo produce IL-10 at the location.

15. A kit for collecting and administering adiponectin, comprising:
a) a syringe having a barrel having an inner wall, a proximal open end and a distal open end, and
b) a cartridge having a barrel having an inner wall defining a housing, an outer wall, a proximal open end and a distal open end, wherein the outer wall of the cartridge is adapted to fit within the inner wall of the syringe, and
i. an adiponectin-binding agent disposed within the housing.

16. A syringe having a barrel having an inner wall, a proximal open end and a distal open end, wherein the barrel contains an effective amount of adiponectin.

17. An apparatus comprising:
a) a syringe comprising a barrel defining a proximal open end, a distal open end, and an inner wall,
b) a porous annulus having an inner radius and an outer radius, the annulus adapted to be received within the inner wall of the syringe and collect APN.

18. The apparatus of claim 17 further comprising:
a. a solid plug having a proximal end and a distal end, and adapted to be received within the inner radius of the porous annulus and having a plurality of legs extending from the distal end thereof.

19. A method comprising the steps of:
a) intradiscally administering an effective amount of a formulation comprising adiponectin (APN) into an intervertebral disc.

20. A method comprising the steps of:
a) epidurally administering an effective amount of a formulation comprising adiponectin (APN) into the vicinity of a nerve root.
